(19) United States
(12) Patent Application Publication
(10) Pub. No.: US 2004/0018941 A1
(43) Pub. Date: Jan. 29, 2004

(54) Fused-benzene derivatives of thiouracil, herbicidal and desiccant compositions contaning them

(75) Inventors: Sandeep Gupta, Concord, OH (US); David A. Pulman, Mentor, OH (US); Taikyun Roh, Highland Heights, OH (US)

Correspondence Address: SUGHRUE MION, PLLC, 2100 Pennsylvania Avenue, NW, Washington, DC, 20037-3213, US

(73) Assignee: ISHIHARA SANGYO KAISHA, LTD.

(21) Appl. No.: 10/196,224

(22) Filed: Jul. 17, 2002

(51) Int. Cl. A01N 43/54; C 07D 4 3/02

(52) U.S. Cl. 504/243; 544/310

(57) ABSTRACT

The invention relates to certain substituted fused-benzene compounds (1), herbicidal compositions containing them, herbicidal methods of use and processes for preparing these compounds;

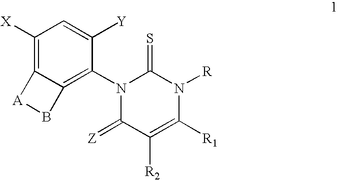

wherein A, B, X, Y, R, R₁, R₂, and Z are as described herein, as well as salts thereof

### BACKGROUND OF THE INVENTION

1. Field of the Invention

The present invention relates to novel fused benzene derivatives of thiouracils, their salts and compositions, intermediates, process for their production, and their use as herbicides.

2. Description of the Related Art

U.S. Pat. No. 4,859,229 discloses the herbicidal utility of uracil derivatives, in which the phenyl ring of the described compounds did not have any 2,6-disubsutitutions. Recently WO97/08170 and WO97/08171 disclosed benzoxazole and benzothiazole derivatives which exhibit herbicidal activity. U.S. Pat. No. 5,169,431 disclosed benzofuran or benzothiophene type derivatives and WO97/29105 disclosed benzofuran derivatives. WO93/14073 described substituted dihydrobenzofuran type compounds, U.S. Pat. No. 5,521,147 disclosed dihydrobenzofuran and dihydrobenzofuran-3-one type derivatives, and WO95/33746 disclosed cyclic sulfonamide derivatives. U.S. Pat. No. 5,346,881 disclosed benzodioxin or benzodioxole derivatives with herbicidal activity and JP 09301973 disclosed 2H-chromene type derivatives. WO97/12886 disclosed benzisoxazole or benzisoxazolidinone derivatives as herbicides and WO97/42188 disclosed indole type compounds with herbicidal activity. Despite the broad coverage of these patents, the general structure of the present invention containing the thiouracil heterocycle has not been described. The specific fused-benzene compounds of the formula 1 mentioned below are not known and are novel. The compounds of present invention exhibit potent herbicidal activity when applied pre or postemergence.

### SUMMARY OF THE INVENTION

The invention delineates a method for the control of undesired vegetation in a plantation crop by the application to the locus of the crop an effective amount of a compound described herein.

The present application describes certain herbicidal fused benzene derivatives of the formula (1) including all geometric and stereo isomers, and their salts, as well as compositions containing them and methods of preparation for these compounds.

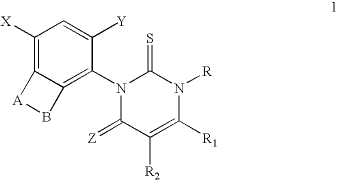

wherein

X is halogen, cyano, nitro, or haloalkyl;

Y is hydrogen or halogen;

Z is oxygen, sulfur or imino;

R is alkyl, haloalkyl or amino;

R₁ is haloalkyl;

R₂ is hydrogen, halogen, nitro, or substituted or unsubstituted amino group;

A is —N═C(R₃)—; —C(R₃)═C(R₄)—; —N(R₃)—C(R₃′)═C(R₄)—; —O—C(R₃)(R₄)—C(R₃′)(R₄′)—; —O—C(R₃)═C(R₄)—; or —C(R₃)═C(R₃′)—C(R₃″)(R₄)—;

B is oxygen or a bond;

R₃ and R can be taken together to represent oxygen, sulfur or an unsubstituted or substituted imino or an oxime group; or R₃, R₃′, R₃″, R₄ and R₄′ are independent of each other and is selected from the group consisting of hydrogen, halogen, hydroxy, mercapto, amino, cyano, nitro, (C1-6)alkyl, (C1-6)haloalkyl, (C1-6)alkoxy,(C1-6)haloalkoxy, (C1-6)alkoxyalkyl, (C2-6)alkynyl, (C2-6)alkenyl, aryl, heteroaryl, aryloxy, heteroaryloxy, (C3-6)cycloalkyl, (C3-6)cycloalkylcarbonyl, carboxy, (C1-6)alkylcarbonyl, arylcarbonyl, (C1-3)haloalkylcarbonyl, (Cl-6)alkylcarbonyloxy, (C1-6)haloalkylcarbonyloxy, (C1-6)alkoxycarbonyl, (C1-6)haloalkoxycarbonyl, (C1-6)alkylthiocarbonyl, (C1-6)haloalkylthiocarbonyl, (C1-6)alkoxythiocarbonyl, (C1-6)haloalkoxytbiocarbonyl, (C1-6)alkylamino, arylsulfonylamino, arylamino, (C1-6)alkylthio, arylthio, (C2-6)alkenylthio, (C2-6)alkynylthio, (C1-6)alkylsulfinyl, (C2-6)alkenylsulfinyl, (C2-6)alkynylsulfinyi, (Ci-6)alkylsulfonyl, (C2-6)alkenylsulfonyl, (C2-6)alkynylsulfonyl, arylsulfonyl, where any of these groups may be unsubstituted or substituted with any of the functional groups represented by one more of the following; halogen, hydroxy, mercapto, cyano, nitro, amino, caboxy, (C1-6)alkyl, (C1-6)haloalkyl, (C1-6)alkylcarbonyl, (C1-6)alkylcarbonyloxy, (C1-6)haloalkylcarbonyl, (C1-6)haloalkylcarbonyloxy, (C1-6)alkoxy, (C1-6)alkoxycarbonyl, aminocarbonyl, (C1-6)alkylaminocarbonyl, (C1-6)haloalkoxy, (C1-6)haloalkoxycarbonyl, (C1-6)alkylsulfonyl, (C1-6)haloalkylsulfonyl, aryl, halooaryl, alkoxyaryl, aryloxy, arylthio, haloaryloxy, heteroaryl, heteroaryloxy, (C3-7)cycloalkyl and other related groups.

Preferred compounds for the reasons of greater herbicidal efficacy are represented by the formula 1 wherein

X is halogen or cyano;

Y is a halogen;

Z is oxygen;

R is (C₁₋₄)alkyl;

R₁ is (C₁₋₄)haloalkyl;

R₂ is hydrogen.

More preferred compounds for the reasons of greater herbicidal efficacy are represented by the formula 1 wherein

X is chlorine;

Y is fluorine;

Z is oxygen;

R is methyl;

R₁ is trifluoromethyl;

R₂ is hydrogen; and/or

-A-B- is —N═C(R₃)—O—; —C(R₃)═C(R₄)—O—; —N(R₃)—C(R₃′)═C(R₄)—; —O—C(R₃)(R₄)—C(R₃′)(R₄′)—O—; —O—C(R₃)═C(R₄)—; or —C(R₃)═C(R₃′)-C(R₃¹¹)(R₄)—O—.

Most preferred compounds for the reasons of greater herbicidal efficacy are represented by the formula 1 wherein

X is chlorine;

Y is fluorine;

Z is oxygen;

R is methyl;

R₁ is trifluoromethyl;

R₂ is hydrogen;

-A-B- is —N═C(R₃)—O—; or —O—C(R₃)═C(R₄)—; and

R₃ and R₄ are independent of each other and may be selected from the group consisting of hydrogen, halogen, cyano, nitro, (C1-6)alkyl, (C1-6)haloalkyl, (C1-6)alkoxy,(C1-6)haloalkoxy, (C1-6)alkoxyalkyl, (C2-6)alkynyl,(C2-6)alkenyl, aryl, heteroaryl, aryloxy, heteroaryloxy, (C3-6)cycloalkyl, (C3-6)cycloalkylcarbonyl, carboxy, (C1-6)alkylcarbonyl, arylcarbonyl, (Cl-3)haloalkylcarbonyl, (C1-6)alkylcarbonyloxy, (C1-6)haloalkylcarbonyloxy, (C1-6)alkoxycarbonyl, (C1-6)haloalkoxycarbonyl, (C1-6)alkylthiocarbonyl, (Cl-6)haloalkylthiocarbonyl, (C1-6)alkoxythiocarbonyl, (C1-6)haloalkoxythiocarbonyl,(C1-6)alkylamino, arylsulfonylamino, arylamino, where any of these groups may be unsubstituted or substituted with any of the functional groups represented by one more of the following; halogen, cyano, nitro, (C1-6)alkyl, (C1-6)haloalkyl, (C1-6)alkylcarbonyl, (C1-6)alkylcarbonyloxy, (C1-6)haloalkylcarbonyl, (C1-6)haloalkylcarbonyloxy, (C1-6)alkoxy, (C1-6)alkoxycarbonyl, aminocarbonyl, (C1-6)alkylaminocarbonyl, (C1-6)haloalkoxy, (C1-6)haloalkoxycarbonyl, (C1-593 6)alkylsulfonyl, (C1-6)haloalkylsulfonyl, aryl, halooaryl, alkoxyaryl, aryloxy, arylthio, haloaryloxy, heteroaryl, heteroaryloxy, (C3-7)cycloalkyl and other related groups.

In the definitions given above, unless the term alkyl, alkenyl, or halogen are defined or mentioned, the term alkyl used either alone or in compound words such as “haloalkyl” or “alkylcarbonyl” includes straight-chain or branched chains containing 1 to 6 carbon atoms. The terms of alkenyl and alkynyl include straight chain or branched alkenes and alkynes respectively containing 2 to 6 carbon atoms. The term halogen either alone or in the compound words such as haloalkyl indicates fluorine, chlorine, bromine, or iodine. Further a haloalkyl is represented by an alkyl partially or fully substituted with halogen atoms which may be same or different. The term or part of the term “aryl” or “heteroaryl” are defined as those monocyclic or fused bicyclic aromatic rings wherein at least one ring satisfies the Huckel rule and contains 04 heteroatoms, examples include phenyl, furyl, furazanyl, thienyl, pyrrolyl, pyrazolyl, oxazolyl, oxadiazolyl, imidazolyl, isoxazolyl, thiazolyl, thiadiazolyl, isothiazolyl, tetrazolyl, pyridinyl, pyrimidinyl, pyridazinyl, pyrazinyl, triazinyl, quinolyl, isoquinolyl, quinoxalinyl, benzofuranyl, 2,3-dihydrobenzofuranyl, isobenzofuranyl, benzothienyl, benzodioxolyl, chromanyl, indolinyl, isoindolyl, naphthyl, thienofuranyl, and purinyl. These rings can be attached through any available carbon or nitrogen, for example when the aromatic ring system is furyl, it can be 2-furyl or 3-furyl, for pyrrolyl, the aromatic ring system is 1-pyrrolyl, 2-pyrrolyl, or 3-pyrrolyl, for naphthyl, the carbobicyclic aromatic ring is 1-naphthyl or 2-naphthyl and for benzofuranyl, the aromatic ring system can be 2-. 3-, 4-, 5-, 6-, or 7-benzofuranyl.

### DETAILED DESCRIPTION OF THE INVENTION

The compounds described by the formula I can be prepared by the procedures as described herein. Using commercially available starting materials or those whose synthesis is known in the art, the compounds of this invention may be prepared using methods described in the following schemes, or using modifications thereof, which are within the scope of the art.

Scheme 1 shows the preparation of the thiouracil 1 starting from aniline 2. This was treated with thiophosgene, with or without an organic solvent, and with or without an organic base, at temperatures from 15 to 150° C. The solvents may be ethyl acetate or toluene and the organic base may be triethylamine. The reaction time is usually from 1-5 hours. The resulting isothiocyanate 3 was reacted at temperatures from −20 to −50° C. with the anion generated from alkyl N-methyl-4,4,4-trifluorocrotonate and sodium hydride in a suitable solvent such as tetrahydrofuran.

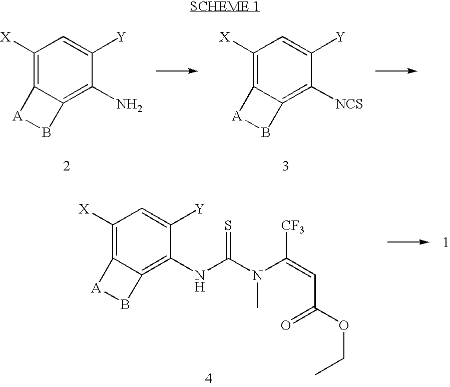

Scheme 2 illustrates the preparation of an useful intermediate for the synthesis of fused-benzene derivatives. The thiouracil 9 may be prepared as shown in Scheme 1. The nitro group may be reduced to amine 10 using either catalytic hydrogenation in a suitable organic solvent such as ethyl acetate or by iron in acetic acid. The ether 10 may be cleaved using boron tribromide in an organic solvent such as methylene chloride at temperatures from 20 to −50° C. over a period of 1-4 hours.

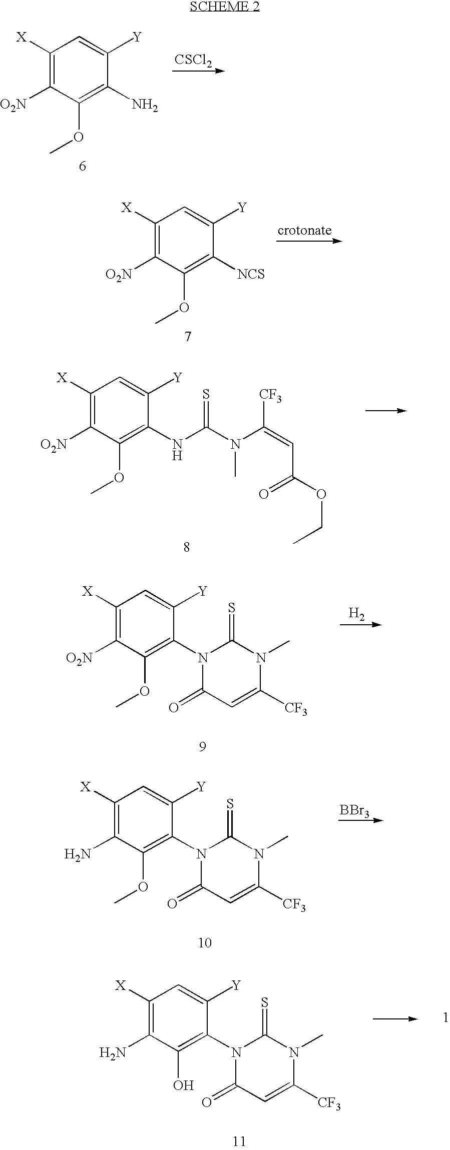

The thiouracil 12 in scheme 3 can be nitrated with nitric acid at a temperature between −10° C. and 30° C. for 0.1-2 hours. The product is obtained by addition of ice-water followed by filtration. 12 can be reduced to the corresponding amine derivative represented by formula 14 by treatment with iron in an acidic medium such as acetic acid or by catalytic hydrogenation. The ether 10 may be cleaved using boron tribromide in an organic solvent such as methylene chloride at temperatures from 20 to −50° C. over a period of 1-4 hours.

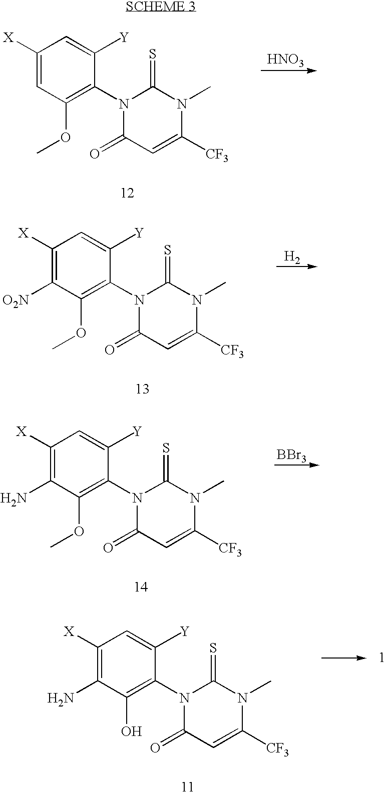

The thiouracil 15 in scheme 4 can be nitrated with nitric acid at a temperature between −10° C. and 30° C. for 0.1-2 hours. The product is obtained by addition of ice-water followed by filtration. 16 can be reduced to the corresponding amine derivative represented by formula 17 by treatment with iron in an acidic medium such as acetic acid or by catalytic hydrogenation. Diazotization of this amine in aqueous sodium nitrite solution in concentrated hydrochloric acid kept at −10 to 5° C. over 15 to 45 minutes yielded the phenol. The ether 10 may be cleaved using boron tribromide in an organic solvent such as methylene chloride at temperatures from 20 to −50° C. over a period of 1-4 hours.

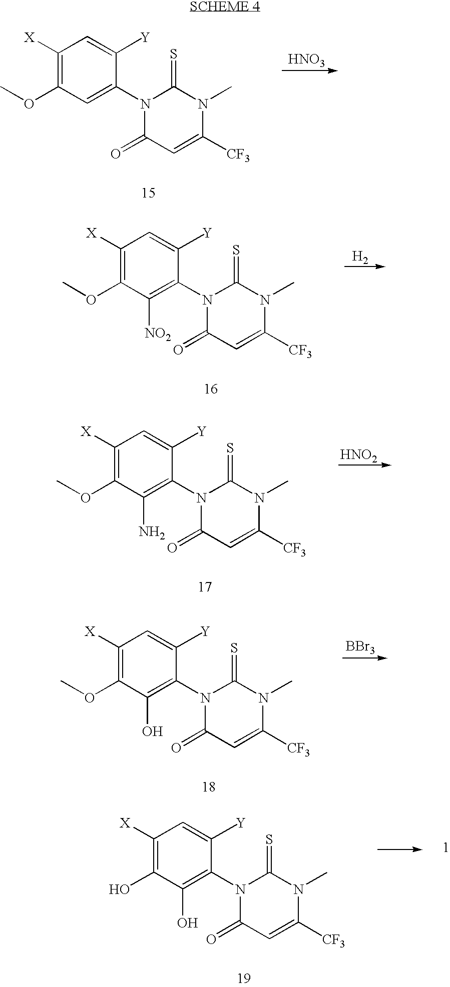

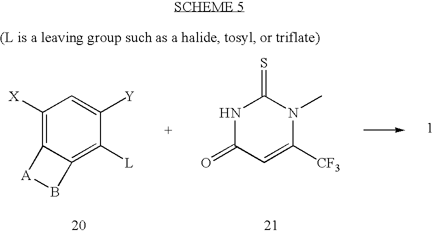

Scheme 6 shows the preparation of a di-phenol 19 using the methodology described in earlier schemes.

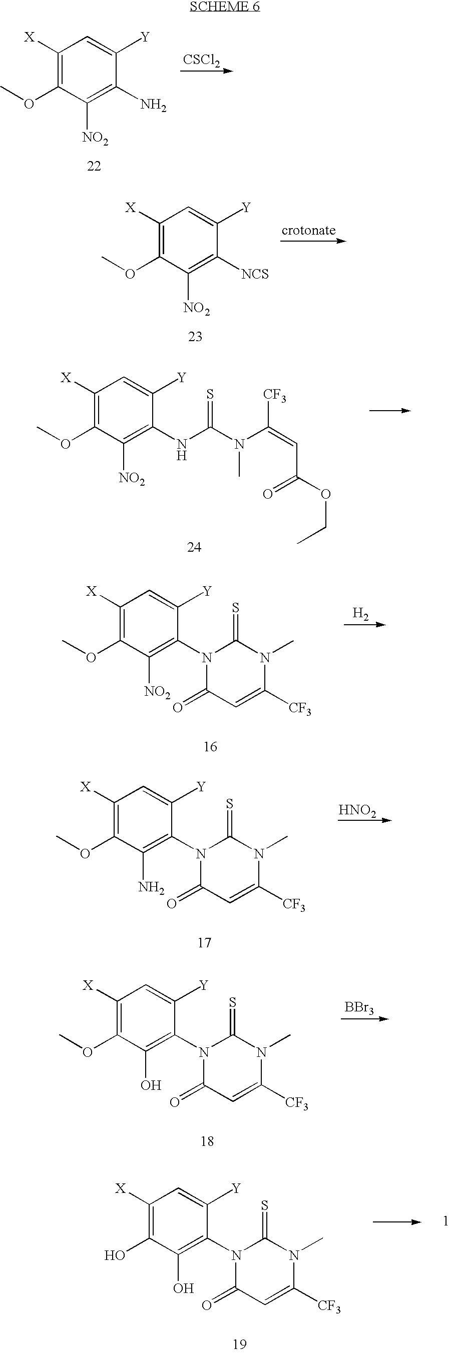

Scheme 7 shows the preparation of a di-phenol 19 using the methodology described in earlier schemes.

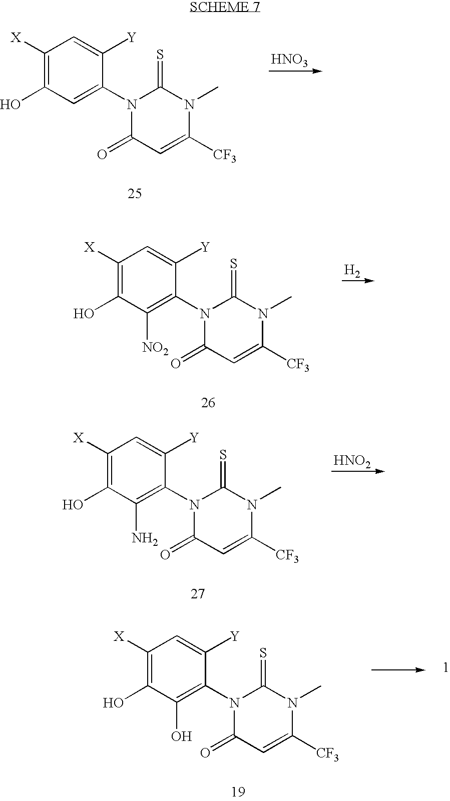

Scheme 8 shows the preparation of an amino-phenol 11 using the methodology described in earlier schemes.

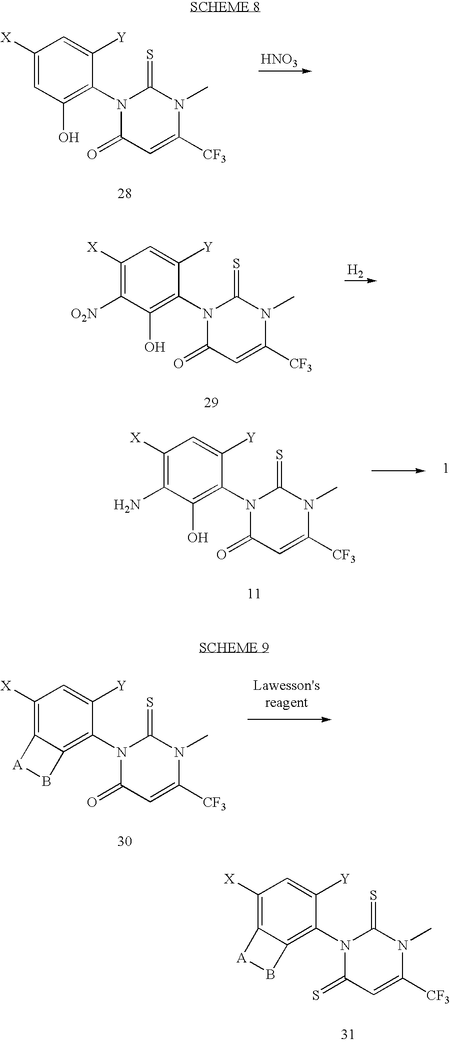

### EXAMPLE I

Preparation of 3-(4-chloro-6-fluoro-1,3-benzoxazol-7-yl)-1-methyl-2-thioxo-6-(trifluoromethyl)-2,3-dihydropyrimidin4(1H)-one (Compound no. 1-1)

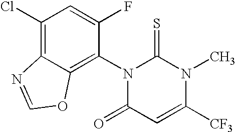

Step 1: Preparation of 1-chloro-5-fluoro4-isothiocyanato-3-methoxy-2-nitrobenzene

4-Chloro-6-fluoro-2-methoxy-3-nitroaniline (6.69 g. 30.3 mmol) was dissolved in toluene (200 ml) and triethylamine (4.61 g, 45.6 mmol) and thiophosgene (5.21 g, 45.3 mmol) was added. Solution was heated under reflux for 2 hr and allowed to cool to room temperature. Solution was passed through a plug of silica gel in toluene and evaporated to afford the title compound (3.7 g, 14.1 mmol). ¹H NMR (CDCl₃, 300 MHz) 4.08 (3H, s), 7.09 (1H, d, J=8.8 Hz) ppm.

Step 2. Preparation of 3-(4-chloro-6-fluoro-2-methoxy-3-nitrophenyl)-1-methyl-2-thioxo-6-(trifluoromethyl)-2,3-dihydropyrimidin4(1H)-one

Ethyl 4,4,4-trifluoro-3-(methylamino)but-2-enoate (3.1 g, 15.3 mmol) in toluene (75 ml) was slowly added to a stirred suspension of sodium hydride (60%, 0.61 g) in anhydrous N,N-dimethylformamide (75 ml) at −10° C. The solution was stirred for 0.5 hr at this temperature and cooled to −50° C. The above 1-chloro-5-fluoro4-isothiocyanato-3-methoxy-2-nitrobenzene (3.64 g, 13.9 mmol) was dissolved in toluene (75 ml) was added drop wise to the stirred solution while maintaining the temperature at −50° C. The solution was allowed to warm to −20° C. and stirred for 2 hr. After neutralization with dilute hydrochloric acid [con. HCl (1.6 ml) in water (50 ml)], the solution was partitioned between water and ethyl acetate, separated, dried (anhydrous sodium sulfate) and the organic layer evaporated to give the crude product. This product was essentially the un-cyclized thiourea derivative (NMR). The residue was dissolved in toluene (100 ml), mixed with triethyl amine (1 ml) and heated under reflux for 0.5 hr. Column chromatography over silica gel (eluent, hexane:ethyl acetate, 90:10) afforded the title compound (3.61 g, 8.7 mmol). ¹H NMR (CDCl₃, 300 MHz) 3.85 (3H, s), 3.93 (3H, m), 6.59 (1H, s), 7.17 (1H, d, J=8.5 Hz) ppm.

Step 3. Preparation of 3-(3-amino-4-chloro-6-fluoro-2-methoxyphenyl) 1-methyl-2-thioxo-6-(trifluoromethyl)-2,3-dihydropyrimidin4(1H)-one

3-(4-Chloro-6-fluoro-2-methoxy-3-nitrophenyl)-1-methyl-2-thioxo-6-(trifluoromethyl)-2,3-dihydropyrimidin4(1H)-one (6.86 g, 16.6 mmol) was dissolved in acetic acid (150 ml) and iron powder (4.63 g, 258.6 mmol) was added. The solution was stirred at ambient temperature under nitrogen atmosphere for 6 hr and water was added. Extraction was carried out with ethyl acetate. Organic layer was washed with water, brine, and dried with anhydrous sodium sulfate followed by evaporation to afford the title compound (5.8 g, 15.1 mmol). ¹H NMR (CDCl₃, 300 MHz) 3.76 (3H, s), 3.93 (3H, s), 4.04 (2H, br s), 6.59 (1H, s), 7.04 (1H, d, J=9.0 Hz) ppm.

Step 4. Preparation of 3-(3-amino-4-chloro-6-fluoro-2-hydroxyphenyl) 1-methyl-2-thioxo-6-(trifluoromethyl)-2,3-dihydropyrimidin4(1H)-one

A solution of 3-(3-amino-4-chloro-6-fluoro-2-methoxyphenyl) 1-methyl-2-thioxo-6-(trifluoromethyl)-2,3-dihydropyrimidin4(1H)-one (4.06 g, 10.6 mmol) and borontribromide (10.6 g, 42.3 mmol) in methylene chloride (250 ml) was stirred at room temperature for 1 hr under a nitrogen atmosphere. The solution was poured into water and extracted with methylene chloride. The organic layer was dried over anhydrous sodium sulfate followed by evaporation to afford the title compound (3.09 g, 8.4 mmol). ¹H NMR (CDCl₃, 300 MHz) 3.92 (3H, m), 6.56 (1H, s), 6.88 (1H, d, J=9.3 Hz) ppm.

Step 5.

Triethyl orthoformate (25 ml) was added to 3-(3-amino-4-chloro-6-fluoro-2-hydroxyphenyl) 1-methyl-2-thioxo-6-(trifluoromethyl)-2,3-dihydropyrimidin-4(1H)-one (3.09 g, 8.4 mmol) and a drop of con. sulfuric acid was added. The resulting mixture was heated at 130° C. under nitrogen atmosphere for 1 hr. Excess reagent was removed under reduced pressure and the product partitioned between water and ethyl acetate. Organic layer was evaporated to furnish a crude product which was purified by column chromatography over silica gel using hexane:ethyl acetate (85:15) as eluent to afford the title compound (2.1 g, 5.5 mmol).

### EXAMPLE 2

Preparation of 3-[2-t-butyl-4-chloro-6-fluoro-1,3-benzoxazol-7-yl]-1-methyl-2-thioxo-6-(trifluoromethyl)-2,3-dihydropyrimidin-4(1H)-one (Compound no. 1-3)

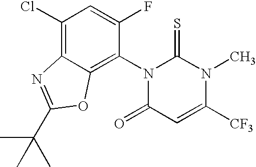

Step 1. 2-t-Butyl-4-chloro-6-fluoro-1,3-benzoxazol-7-amine

Ethyl N-(2-t-butyl-4-chloro-6-fluorobenzoxazol-7-yl)carbamate (WO 97/08170) (5.0 g) and potassium hydroxide (1.5 g) dissolved in ethylene glycol (50 ml) were stirred and heated at 150° C. for 3 hr. The solution was cooled, acidified with dilute hydrochloric acid and extracted into ethyl acetate. It was dried over anhydrous sodium sulfate, evaporated under reduced pressure and chromatographed on silica eluting with 5% methanol in methylene chloride. The product was isolated as a white solid (2.94 g). ¹H NMR (CDCl₃, 300 MHz) 1.50 (9H, s), 3.93 (2H, br s), 7.05 (1H, d, J=11.0 Hz).

Step 2. 2-t-Butyl-4-chloro-6-fluoro-1,3-benzoxazol-7-isocyanate

The above amine (1.02 g) and triethylamine (1.27 g) dissolved in dry ethyl acetate was added drop-wise under nitrogen to a solution of thiophosgene (1.47 g) in dry ethyl acetate stirred at 0° C. The mixture was heated at reflux for 2 hr, cooled, passed through a plug of silica gel and evaporated to give 2-t-butyl-4-chloro-6-fluoro-1,3-benzoxazol-7-isocyanate (1.1 g). ¹H NMR (CDCl₃, 300 MHz) 1.52 (9H, s), 7.17 (1H, d, J=10.0 Hz).

Step 3.

Ethyl 4,4,4-trifluoro-3-(methylamino)but-2-enoate (0.85 g) in toluene (10 ml) was slowly added to a stirred suspension of sodium hydride (60%, 0.17 g) in anhydrous N,N-dimethylformamide (10 ml) at −10° C. The solution was stirred for 0.5 hr at this temperature and cooled to −50° C. The above 2-t-butyl-4-chloro-6-fluoro-1,3-benzoxazol-7-isocyanate (1.1 g) dissolved in toluene (10 ml) and N,N-dimethyl-formamide (10 ml), was added drop-wise to the stirred solution while maintaining the temperature at −50° C. The solution was allowed to warm to −30° C. and stirred for 2 hr. After neutralization with dilute hydrochloric acid (1.0 ml cone. HCl in water 10 ml), the solution was partitioned between water and ethyl acetate, separated, dried (anhydrous sodium sulfate) and the organic layer evaporated to give the crude product. Column chromatography over silica gel (eluent, hexane:ethyl acetate, 90:10) afforded the title compound (0.67 g).

### EXAMPLE 3

Preparation of 3-(7-chloro-5-fluoro-2-methyl-1-benzofuran-4-yl)-1-methyl-2-thioxo-6-(trifluoromethyl)-2,3-dihydropyrimidin-4(1H)-one (Compound no. 2-2)

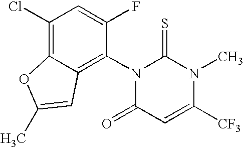

Step 1. 7-Chloro-5-fluoro4-isothiocyanato-2-methyl-1-benzofuran

7-Chloro-5-fluoro-2-methyl-1-benzofuran-4-amine (1.1 g) and triethylamine (1.0 g) dissolved in dry ethyl acetate was added drop-wise under nitrogen to a solution of thiophosgene (1.4 g) in dry ethyl acetate stirred at 0° C. The mixture was heated at reflux for 2 h, cooled, passed through a plug of silica sel and evaporated to give 7-chloro-5-fluoro4-isothiocyanato-2-methyl-1-benzofuran (1.2 g). ¹H NMR (CDCl₃, 300 MHz) 2.51 (3H, s), 6.57 (1H, s), 7.04 (1H, d, J=9.8 Hz).

Step 2.3-(7-Chloro-5-fluoro-2-methyl-1-benzofuran-4-yl)-1-methyl-2-thioxo-6-(trifluoromethyl)-2,3-dihydronprimidine-4(H)-one

Ethyl 4,4,4-trifluoro-3-(methylamino)but-2-enoate (0.90 g) in toluene (10 ml) was slowly added to a stirred suspension of sodium hydride (60%, 0.19 g) in anhydrous N,N-dimethylformamide (10 ml) at −10° C. The solution was stirred for 0.5 hr at this temperature and cooled to −50° C. The above 7-chloro-5-fluoro4-isothiocyanato-2-methyl-1-benzofuran (1.2 g) dissolved in toluene (10 ml) and N,N-dimethyl-formamide (10 ml), was added drop-wise to the stirred solution while maintaining the temperature at −50° C. The solution was allowed to warm to −30° C. and stirred for 2 hr. After neutralization with dilute hydrochloric acid (1.0 ml cone. HCl in water 10 ml), the solution was partitioned between water and ethyl acetate, separated, dried (anhydrous sodium sulfate) and the organic layer evaporated to give the crude product. Column chromatography over silica gel (eluent, hexane:ethyl acetate, 90:10) afforded the title compound (0.71 g). ¹H NMR (CDCl₃, 300 MHz) 2.47 (3H, s), 3.93 (3H, s), 6.25 (1H, s), 6.60 (1H, s), 7.15 (1H, d, J=9.75 Hz)

Using the procedures as described in Schemes 1-9 and Examples 1-3, the compounds of this invention can be readily prepared. Tables 1-6 list structures for few representative compounds of this invention.

TABLE 1

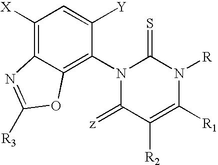

| Cmpd. No. | X | Y | R | R₁ | R₂ | R₃ | Z |
|---|---|---|---|---|---|---|---|
| 1-1 | Cl | F | Me | CF₃ | H | H | O |
| 1-2 | Cl | F | Me | CF₃ | H | Me | O |
| 1-3 | Cl | F | Me | CF₃ | H | t-Bu | O |
| 1-4 | Cl | H | Me | CF₃ | H | t-Bu | O |
| 1-5 | H | F | Me | CF₃ | H | t-Bu | O |
| 1-6 | Cl | F | Me | CF₃ | H | Et | O |
| 1-7 | Cl | F | Me | CF₃ | H | Pr | O |
| 1-8 | Cl | F | Me | CF₃ | H | i-Pr | O |
| 1-9 | Cl | F | Me | CF₃ | H | n-Bu | O |
| 1-10 | CN | F | Me | CF₃ | H | Me | O |
| 1-11 | Cl | F | Me | CF₃ | H | —C(CH₃)═CH₂ | O |
| 1-12 | Cl | F | Me | CF₃ | H | 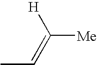 | O |
| 1-13 | Cl | F | Me | CF₃ | H | —CH═C(CH₃)₂ | O |
| 1-14 | H | H | Me | CF₃ | H | t-Bu | O |
| 1-15 | Cl | F | Me | CF₃ | H | t-Bu | S |
| 1-16 | Br | F | Me | CF₃ | H | t-Bu | O |
| 1-17 | CN | F | Me | CF₃ | H | t-Bu | O |
| 1-18 | C | F | Me | CF₃ | H | Ph | O |
| 1-19 | Cl | F | Me | CF₃ | H | 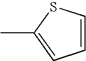 | O |
| 1-20 | Cl | F | Me | CF₃ | H | 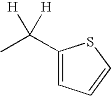 | O |
| 1-21 | Cl | F | Me | CF₃ | H | 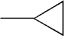 | O |
| 1-22 | Cl | F | Me | CF₃ | H | 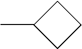 | O |
| 1-23 | Cl | F | Me | CF₃ | H | —CH₂CH₂Ph | O |
| 1-24 | Cl | F | Me | CF₃ | H | 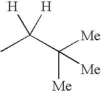 | O |
| 1-25 | CI | F | Me | CF₃ | H | 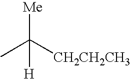 | O |
| 1-26 | Cl | F | Me | CF₃ | H | 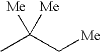 | O |
| 1-27 | Cl | F | Me | CF₃ | H | 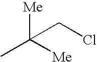 | O |
| 1-28 | Cl | F | Me | CF₃ | H | 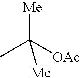 | O |
| 1-29 | Cl | F | Me | CF₃ | H | 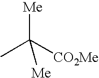 | O |
| 1-30 | Cl | F | Me | CF₃ | H | 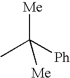 | O |
| 1-31 | Cl | F | Me | CF₃ | H | 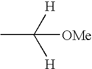 | O |
| 1-32 | Cl | F | Me | CF₃ | H | 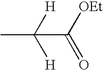 | O |
| 1-33 | Cl | F | Me | CF₃ | H | —CH₂Cl | O |
| 1-34 | Cl | F | Me | CF₃ | H | 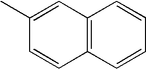 | O |
| 1-35 | Cl | F | Me | CF₃ | H | 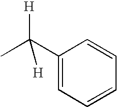 | O |
| 1-36 | Cl | F | Me | CF₃ | H | 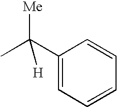 | O |
| 1-37 | Cl | F | Me | CF₃ | H | 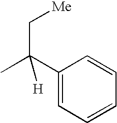 | O |
| 1-38 | Cl | F | Me | CF₃ | H | —CH₂Cl | O |
| 1-39 | Cl | F | Me | CF₃ | H | —CH₂CN | O |
| 1-40 | Cl | F | Me | CF₃ | H | 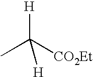 | O |
| 1-41 | Cl | F | Me | CF₃ | H | 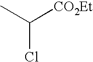 | O |
| 1-42 | Cl | F | Me | CF₃ | H | CO₂Me | O |
| 1-43 | Cl | F | Me | CF₃ | H | —CH₂OPh | O |
| 1-44 | Cl | F | Me | CF₃ | H | 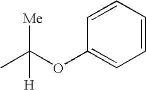 | O |
| 1-45 | Cl | F | Me | CF₃ | H | 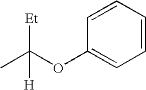 | O |
| 1-46 | Cl | F | Me | CF₃ | H | 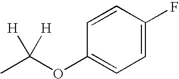 | O |
| 1-47 | Cl | F | Me | CF₃ | H | 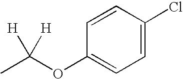 | O |
| 1-48 | Cl | F | Me | CF₃ | H | Cl | O |
| 1-49 | Cl | F | Me | CF₃ | H | —OMe | O |
| 1-50 | Cl | F | Me | CF₃ | H | —OPh | O |
| 1-51 | Cl | F | Me | CF₃ | H | NH2 | O |
| 1-52 | Cl | F | Me | CF₃ | H | —N(Et)₂ | O |
| 1-53 | Cl | F | Me | CF₃ | H | | O |
| 1-54 | Cl | F | Me | CF₃ | H | —SH | O |
| 1-55 | Cl | F | Me | CF₃ | H | —SMe | O |
| 1-56 | Cl | F | Me | CF₃ | H | 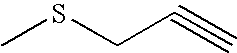 | O |
| 1-57 | Cl | F | Me | CF₃ | H | 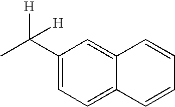 | O |
| 1-58 | Cl | F | Me | CF₃ | H | 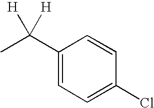 | O |
| 1-59 | Cl | F | Me | CF₃ | H | 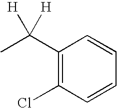 | O |
| 1-60 | Cl | F | Me | CF₃ | H | 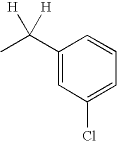 | O |
| 1-61 | Cl | F | Me | CF₃ | H | 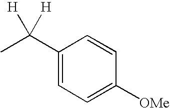 | O |
| 1-62 | Cl | F | Me | CF₃ | H | 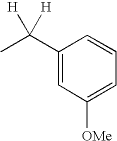 | O |
| 1-63 | Cl | F | Me | CF₃ | H | 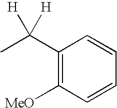 | O |
| 1-64 | Cl | F | Me | CF₃ | H | 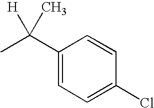 | O |
| 1-65 | Cl | F | Me | CF₃ | H | 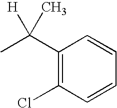 | O |
| 1-66 | Cl | F | Me | CF₃ | H | 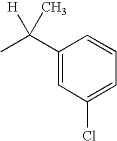 | O |
| 1-67 | Cl | F | Me | CF₃ | H | 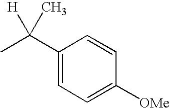 | O |
| 1-68 | Cl | F | Me | CF₃ | H | 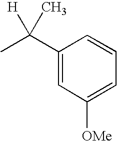 | O |
| 1-69 | Cl | F | Me | CF₃ | H | 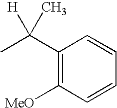 | O |
| 1-70 | Cl | F | Me | CF₃ | H | 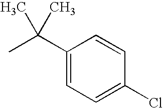 | O |
| 1-71 | Cl | F | Me | CF₃ | H | 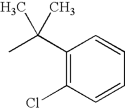 | O |
| 1-72 | Cl | F | Me | CF₃ | H | 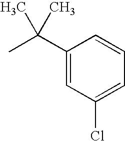 | O |
| 1-73 | Cl | F | Me | CF₃ | H | 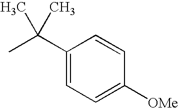 | O |
| 1-74 | Cl | F | Me | CF₃ | H | 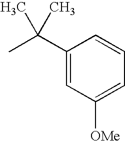 | O |
| 1-75 | Cl | F | Me | CF₃ | H | 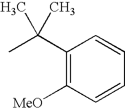 | O |
| 1-76 | Cl | F | Me | CF₃ | H | CN | O |
| 1-77 | Cl | F | Me | CF₃ | H | —CH₂—C≡CH | O |
| 1-78 | Cl | F | Me | CF₃ | H | 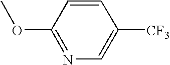 | O |
| 1-79 | Cl | F | Me | CF₃ | H | CF₃ | O |

TABLE 2

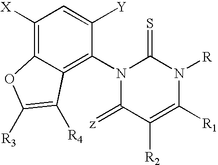

| Cmpd. No. | X | Y | R | R₁ | R₂ | R₃ | R₄ | Z |
|---|---|---|---|---|---|---|---|---|
| 2-1 | Cl | F | Me | CF₃ | H | H | H | O |
| 2-2 | Cl | F | Me | CF₃ | H | Me | H | O |
| 2-3 | Cl | F | Me | CF₃ | H | t-Bu | H | O |
| 2-4 | Cl | H | Me | CF₃ | H | t-Bu | H | O |
| 2-5 | Cl | Cl | Me | CF₃ | H | t-Bu | H | O |
| 2-6 | Cl | F | Me | CF₃ | H | Et | H | O |
| 2-7 | Cl | F | Me | CF₃ | H | Pr | H | O |
| 2-8 | Cl | F | Me | CF₃ | H | i-Pr | H | O |
| 2-9 | Cl | F | Me | CF₃ | H | n-Bu | H | O |
| 2-10 | CN | F | Me | CF₃ | H | Me | H | O |
| 2-11 | Cl | F | Me | CF₃ | H | —C(CH₃)═CH₂ | H | O |
| 2-12 | Cl | F | Me | CF₃ | H | 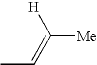 | H | O |
| 2-13 | Cl | F | Me | CF₃ | H | —CH═C(CH₃)₂ | H | O |
| 2-14 | Cl | F | Me | CF₃ | H | i-Pr | H | O |
| 2-15 | H | H | Me | CF₃ | H | t-Bu | H | O |
| 2-16 | CN | F | Me | CF₃ | H | t-Bu | H | O |
| 2-17 | CN | H | Me | CF₃ | H | t-Bu | H | S |
| 2-18 | Cl | F | Me | CF₃ | H | Ph | H | O |
| 2-19 | Cl | F | Me | CF₃ | H | —CH₂CH₂Ph | H | O |
| 2-20 | Cl | F | Me | CF₃ | H | 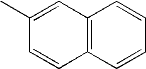 | H | O |
| 2-21 | Cl | F | Me | CF₃ | H | —CH₂Ph | H | O |
| 2-22 | Cl | F | Me | CF₃ | H | —CH(CH₃)Ph | H | O |
| 2-23 | Cl | F | Me | CF₃ | H | —CH₂Cl | H | O |
| 2-24 | Cl | F | Me | CF₃ | H | —CH₂CN | H | O |
| 2-25 | Cl | F | Me | CF₃ | H | —CH₂OPh | H | O |
| 2-26 | Cl | F | Me | CF₃ | H | Cl | H | O |
| 2-27 | Cl | F | Me | CF₃ | H | —OMe | H | O |
| 2-28 | Cl | F | Me | CF₃ | H | —OPh | H | O |
| 2-29 | Cl | F | Me | CF₃ | H | NH₂ | H | O |
| 2-30 | Cl | F | Me | CF₃ | H | —N(Et)₂ | H | O |
| 2-31 | Cl | F | Me | CF₃ | H | —SMe | H | O |
| 2-32 | Cl | F | Me | CF₃ | H | t-Bu | H | S |
| 2-33 | CN | F | Me | CF₃ | H | t-Bu | H | S |
| 2-34 | Cl | F | Me | CF₃ | H | t-Bu | H | S |
| 2-35 | Cl | F | Me | CF₃ | H | CN | H | O |
| 2-36 | Cl | F | Me | CF₃ | I | Me | H | O |
| 2-37 | Cl | F | Me | CF₃ | H | Me | H | S |
| 2-38 | Cl | F | Me | CF₃ | I | Me | H | S |
| 2-39 | Cl | F | Me | CF₃ | H | CF₃ | H | O |

TABLE 3

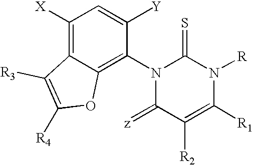

| Cmpd. No. | X | Y | R | R₁ | R₂ | R₃ | R₄ | Z |
|---|---|---|---|---|---|---|---|---|
| 3-1 | Cl | F | Me | CF₃ | H | H | H | O |
| 3-2 | Cl | F | Me | CF₃ | H | Me | Me | O |
| 3-3 | Cl | F | Me | CF₃ | H | Me | t-Bu | O |
| 3-4 | Cl | H | Me | CF₃ | H | Me | t-Bu | O |
| 3-5 | Cl | Cl | Me | CF₃ | H | Me | t-Bu | O |
| 3-6 | Cl | F | Me | CF₃ | H | Me | Et | O |
| 3-7 | Cl | F | Me | CF₃ | H | Me | Pr | O |
| 3-8 | Cl | F | Me | CF₃ | H | Me | i-Pr | O |
| 3-9 | Cl | F | Me | CF₃ | H | Me | n-Bu | O |
| 3-10 | CN | F | Me | CF₃ | H | Me | Me | O |
| 3-11 | Cl | F | Me | CF₃ | H | Me | —C(CH₃)═CH₂ | O |
| 3-12 | Cl | F | Me | CF₃ | H | Me | 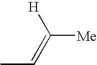 | O |
| 3-13 | Cl | F | Me | CF₃ | H | Me | —CH═C(CH₃)₂ | O |
| 3-14 | Cl | F | Me | CF₃ | H | Me | i-Pr | O |
| 3-15 | H | H | Me | CF₃ | H | Me | t-Bu | O |
| 3-16 | CN | F | Me | CF₃ | H | Me | t-Bu | O |
| 3-17 | CN | H | Me | CF₃ | H | Me | t-Bu | S |
| 3-18 | Cl | F | Me | CF₃ | H | Me | Ph | O |
| 3-19 | Cl | F | Me | CF₃ | H | Me | —CH₂CH₂Ph | O |
| 3-20 | Cl | F | Me | CF₃ | H | Me | 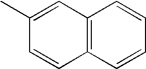 | O |
| 3-21 | Cl | F | Me | CF₃ | H | Me | —CH₂Ph | O |
| 3-22 | Cl | F | Me | CF₃ | H | Me | —CH(CH₃)Ph | O |
| 3-23 | Cl | F | Me | CF₃ | H | Me | —CH₂Cl | O |
| 3-24 | Cl | F | Me | CF₃ | H | Me | —CH₂CN | O |
| 3-25 | Cl | F | Me | CF₃ | H | Me | —CH₂OPh | O |
| 3-26 | Cl | F | Me | CF₃ | H | Me | Cl | O |
| 3-27 | Cl | F | Me | CF₃ | H | Me | —OMe | O |
| 3-28 | Cl | F | Me | CF₃ | H | Me | —OPh | O |
| 3-29 | Cl | F | Me | CF₃ | H | Me | NH₂ | O |
| 3-30 | Cl | F | Me | CF₃ | H | Me | —N(Et)₂ | O |
| 3-31 | Cl | F | Me | CF₃ | H | Me | —SMe | O |
| 3-32 | Cl | F | Me | CF₃ | H | Me | t-Bu | S |
| 3-33 | Cl | F | Me | CF₃ | H | Me | t-Bu | O |
| 3-34 | Cl | F | Me | CF₃ | I | Me | Me | O |
| 3-35 | Cl | F | Me | CF₃ | H | Cl | Me | S |
| 3-36 | Cl | F | Me | CF₃ | H | Me | Me | O |
| 3-37 | Cl | F | Me | CF₃ | H | Me | CF₃ | O |

TABLE 4

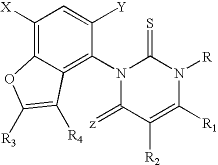

| Cmpd. No. | X | Y | R | R₁ | R₂ | R₃ | R₄ | Z |
|---|---|---|---|---|---|---|---|---|
| 4-1 | Cl | F | Me | CF₃ | H | H | H | O |
| 4-2 | Cl | F | Me | CF₃ | H | Me | H | O |
| 4-3 | Cl | F | Me | CF₃ | H | t-Bu | H | O |
| 4-4 | Cl | H | Me | CF₃ | H | t-Bu | H | O |
| 4-5 | Cl | Cl | Me | CF₃ | H | t-Bu | H | O |
| 4-6 | Cl | F | Me | CF₃ | H | Et | H | O |
| 4-7 | Cl | F | Me | CF₃ | H | Pr | H | O |
| 4-8 | Cl | F | Me | CF₃ | H | i-Pr | H | O |
| 4-9 | Cl | F | Me | CF₃ | H | n-Bu | H | O |
| 4-10 | CN | F | Me | CF₃ | H | Me | H | O |
| 4-11 | Cl | F | Me | CF₃ | H | —C(CH₃)═CH₂ | H | O |
| 4-12 | Cl | F | Me | CF₃ | H | 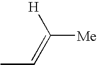 | H | O |
| 4-13 | Cl | F | Me | CF₃ | H | —CH═C(CH₃)₂ | H | O |
| 4-14 | Cl | F | Me | CF₃ | H | i-Pr | H | O |
| 4-15 | H | H | Me | CF₃ | H | t-Bu | H | O |
| 4-16 | CN | F | Me | CF₃ | H | t-Bu | H | O |
| 4-17 | CN | H | Me | CF₃ | H | t-Bu | H | S |
| 4-18 | Cl | F | Me | CF₃ | H | Ph | H | O |
| 4-19 | Cl | F | Me | CF₃ | H | —CH₂CH₂Ph | H | O |
| 4-20 | Cl | F | Me | CF₃ | H | 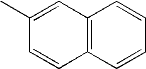 | H | O |
| 4-21 | Cl | F | Me | CF₃ | H | —CH₂Ph | H | O |
| 4-22 | Cl | F | Me | CF₃ | H | —CH(CH₃)Ph | H | O |
| 4-23 | Cl | F | Me | CF₃ | H | —CH₂Cl | H | O |
| 4-24 | Cl | F | Me | CF₃ | H | —CH₂CN | H | O |
| 4-25 | Cl | F | Me | CF₃ | H | —CH₂OPh | H | O |
| 4-26 | Cl | F | Me | CF₃ | H | Cl | H | O |
| 4-27 | Cl | F | Me | CF₃ | H | —OMe | H | O |
| 4-28 | Cl | F | Me | CF₃ | H | —OPh | H | O |
| 4-29 | Cl | F | Me | CF₃ | H | NH₂ | H | O |
| 4-30 | Cl | F | Me | CF₃ | H | —N(Et)₂ | H | O |
| 4-31 | Cl | F | Me | CF₃ | H | —SMe | H | O |
| 4-32 | Cl | F | Me | CF₃ | H | t-Bu | H | S |
| 4-33 | Cl | F | Me | CF₃ | H | t-Bu | H | O |
| 4-34 | Cl | F | Me | CF₃ | H | CN | H | O |
| 4-35 | Cl | F | Me | CF₃ | H | Me | H | S |
| 4-36 | Cl | F | Me | CF₃ | I | Me | H | O |
| 4-37 | Cl | F | Me | CF₃ | H | CF₃ | H | O |

TABLE 5

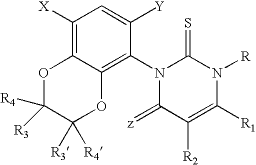

| Cmpd. No. | X | Y | R | R₁ | R₂ | R₃ | R₄ | R₃′ | R₄′ | Z |
|---|---|---|---|---|---|---|---|---|---|---|
| 5-1 | Cl | F | Me | CF₃ | H | H | H | H | H | O |
| 5-2 | Cl | F | Me | CF₃ | H | Me | Me | H | H | O |
| 5-3 | Cl | F | Me | CF₃ | H | t-Bu | H | H | H | O |
| 5-4 | Cl | H | Me | CF₃ | H | t-Bu | H | H | H | O |
| 5-5 | Cl | Cl | Me | CF₃ | H | t-Bu | H | H | H | O |
| 5-6 | Cl | F | Me | CF₃ | H | Et | H | H | H | O |
| 5-7 | Cl | F | Me | CF₃ | H | Pr | H | H | H | O |
| 5-8 | Cl | F | Me | CF₃ | H | i-Pr | H | H | H | O |
| 5-9 | Cl | F | Me | CF₃ | H | n-Bu | H | H | H | O |
| 5-10 | CN | F | Me | CF₃ | H | Me | H | H | H | O |
| 5-11 | Cl | F | Me | CF₃ | H | —C(CH₃)═CH₂ | H | H | H | O |
| 5-12 | Cl | F | Me | CF₃ | H | 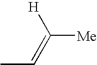 | H | H | H | O |
| 5-13 | Cl | F | Me | CF₃ | H | —CH═C(CH₃)₂ | H | H | H | O |
| 5-14 | Cl | F | Me | CF₃ | H | i-Pr | H | H | H | O |
| 5-15 | H | H | Me | CF₃ | H | t-Bu | H | H | H | O |
| 5-16 | CN | F | Me | CF₃ | H | t-Bu | H | H | H | O |
| 5-17 | CN | H | Me | CF₃ | H | t-Bu | H | H | H | S |
| 5-18 | Cl | F | Me | CF₃ | H | Ph | H | H | H | O |
| 5-19 | Cl | F | Me | CF₃ | H | —CH₂CH₂Ph | H | H | H | O |
| 5-20 | Cl | F | Me | CF₃ | H | 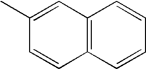 | H | H | H | O |
| 5-21 | Cl | F | Me | CF₃ | H | —CH₂Ph | H | H | H | O |
| 5-22 | Cl | F | Me | CF₃ | H | —CH(CH₃)Ph | H | H | H | O |
| 5-23 | Cl | F | Me | CF₃ | H | —CH₂Cl | H | H | H | O |
| 5-24 | Cl | F | Me | CF₃ | H | —CH₂CN | H | H | H | O |
| 5-25 | Cl | F | Me | CF₃ | H | —CH₂OPh | H | H | H | O |
| 5-26 | Cl | F | Me | CF₃ | H | Cl | H | H | H | O |
| 5-27 | Cl | F | Me | CF₃ | H | —OMe | H | H | H | O |
| 5-28 | Cl | F | Me | CF₃ | H | —OPh | H | H | H | O |
| 5-29 | Cl | F | Me | CF₃ | H | NH2 | H | H | H | O |
| 5-30 | Cl | F | Me | CF₃ | H | —N(Et)₂ | H | H | H | O |
| 5-31 | Cl | F | Me | CF₃ | H | —SMe | H | H | H | O |
| 5-32 | Cl | F | Me | CF₃ | H | t-Bu | H | H | H | S |
| 5-33 | Cl | F | Me | CF₃ | I | t-Bu | H | H | H | O |
| 5-34 | Cl | F | Me | CF₃ | H | CN | H | H | H | O |
| 5-35 | Cl | F | Me | CF₃ | H | Me | H | H | H | O |
| 5-36 | Cl | F | Me | CF₃ | H | Me | H | H | H | S |
| 5-37 | Cl | F | Me | CF₃ | I | Me | H | H | H | O |
| 5-38 | Cl | F | Me | CF₃ | H | CF₃ | H | H | H | O |

TABLE 6

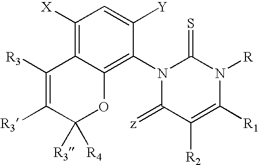

| Cmpd. No. | X | Y | R | R₁ | R₂ | R₃ | R₃′ | R₃″ | R₄ | Z |
|---|---|---|---|---|---|---|---|---|---|---|
| 6-1 | Cl | F | Me | CF₃ | H | H | H | H | H | O |
| 6-2 | Cl | F | Me | CF₃ | H | H | H | Me | Me | O |
| 6-3 | Cl | F | Me | CF₃ | H | H | H | t-Bu | H | O |
| 6-4 | Cl | H | Me | CF₃ | H | H | H | Me | H | O |
| 6-5 | Cl | Cl | Me | CF₃ | H | H | H | Me | H | O |
| 6-6 | Cl | F | Me | CF₃ | H | H | H | Et | H | O |
| 6-7 | Cl | F | Me | CF₃ | H | H | H | Pr | H | O |
| 6-8 | Cl | F | Me | CF₃ | H | H | H | i-Pr | H | O |
| 6-9 | Cl | F | Me | CF₃ | H | H | H | n-Bu | H | O |
| 6-10 | CN | F | Me | CF₃ | H | H | H | Me | H | O |
| 6-11 | Cl | F | Me | CF₃ | H | H | H | O | | O |
| 6-12 | Cl | F | Me | CF₃ | H | H | H | i-Pr | H | O |
| 6-13 | H | H | Me | CF₃ | H | H | H | t-Bu | H | O |
| 6-14 | CN | F | Me | CF₃ | H | H | H | t-Bu | H | O |
| 6-15 | CN | H | Me | CF₃ | H | H | H | t-Bu | H | S |
| 6-16 | Cl | F | Me | CF₃ | H | H | Ph | H | O | |
| 6-17 | Cl | F | Me | CF₃ | H | H | H | —CH₂CH₂Ph | H | O |
| 6-18 | Cl | F | Me | CF₃ | H | H | H | 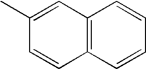 | H | O |
| 6-19 | Cl | F | Me | CF₃ | H | H | H | Cl | H | O |
| 6-20 | Cl | F | Me | CF₃ | H | H | H | —OMe | H | O |
| 6-21 | Cl | F | Me | CF₃ | H | H | H | —OPh | H | O |
| 6-22 | Cl | F | Me | CF₃ | H | H | H | NH2 | H | O |
| 6-23 | Cl | F | Me | CF₃ | H | H | H | —SMe | H | O |
| 6-24 | Cl | F | Me | CF₃ | H | H | H | t-Bu | H | S |
| 6-25 | Cl | F | Me | CF₃ | I | H | H | t-Bu | H | O |
| 6-26 | Cl | F | Me | CF₃ | H | H | H | CN | H | O |
| 6-27 | Cl | F | Me | CF₃ | H | H | H | Me | H | O |
| 6-28 | Cl | F | Me | CF₃ | H | H | H | Me | H | S |
| 6-29 | Cl | F | Me | CF₃ | I | H | H | Me | H | O |
| 6-30 | Cl | F | Me | CF₃ | H | H | H | CF₃ | H | O |

Table 7 lists some of the characterization data for a few representative compounds of this invention.

TABLE 7

| Compd No | ¹H-NMR(300 MHz, ppm) |
|---|---|
| 1-1 | 3.94 (3H, m), 6.62 (1H, s), 7.37 (1H, d, J=10.0 Hz), |
| | 8.08 (1H, s) |
| 1-2 | 2.64 (3H, s), 3.94 (3H, m), 6.62 (1H, s), 7.28 |
| | (1H, d, j=10.5 Hz) |
| 1-3 | 1.47 (9H, s), 3.95 (3H, s), 6.62 (1H, s), 7.26 |
| | (1H, d, J=10.0 Hz) |
| 1-4 | 1.47 (9H, s), 3.94 (3H, s), 6.63 (1H, s), 7.07 |
| | (1H, d, J=8.5 Hz), 7.41 (1H, d, J=8.5 Hz) |
| 1-5 | 1.46 (9H, s), 3.96 (3H, s), 6.63 (1H, s), 7.10 |
| | (1H, m), 7.72 (1H, m) |
| 2-2 | 2.47 (3H, s), 3.93 (3H, s), 6.25 (1H, s), 6.60 |
| | (1H, s), 7.15 (1H, d, J=9.75 Hz) |

### Herbicidal Activity

The compounds of the present invention exhibit excellent herbicidal effects when used as an active ingredient of a herbicide. The herbicide can be used for a wide range of applications, for example on crop lands such as paddy fields, upland farms, orchards and mulberry fields, and non-crop lands such as forests, turf, rights of way, roadsides, farm roads, playgrounds, and factory sites. The application method may be suitably selected for soil treatment application and foliar application.

The compounds of the present invention are capable of controlling noxious weeds including grass (gramineae) such as barnyardgrass (Echinochloa crus-galli), large crabgrass (Digitaria sanguinalis), green foxtail (Setaria viridis), goosegrass (Eleusine indica L.), wild oat (Avena fatua L.), Johnsongrass (Sorghum halepense), quackgrass (Agropyron repens), alexandergrass (Brachiaria plantaginea), paragrass (Panicum purpurascen), sprangletop (Leptochloa chinensis) and red sprangletop (Leptochloa panicea); sedges (or Cyperaceae) such as rice flatsedge (Cyperus iria L.), purple nutsedge (Cyperus rotundus L.), Japanese bulrush (Scirpus Juncoides), flatsedge (Cyperus serotinus), small-flower umbrellaplant (Cyperus difformis), slender spikerush (Eleocharis acicularis), and water chestnut (Eleocharis kuroguwai); alismataceae such as Japanese ribbon wapato (Sagittaria pygmaea), arrow-head (Sagittaria trifolia) and narrowleaf waterplantain (Alisma canaliculatum); pontederiaceae such as monochoria (Monochoria vaginalis) and monochoria species (Monochoria korsakowii); scrophulariaceae such as false pimpernel (Lindernlia pyxidaria) and abunome (Dopatrium Junceum); lythraceae such as tootheup (Rotala indica) and red stem (Ammannia multiflora); and broadleaves such as redroot pigweed (Amaranthus retroflexus), velvetleaf (Abutilon theophrasti), morningglory (Ipomoea hederacea), lambsquarters (Chenopodium album), prickly sida (Sida spinosa L.), common purslane (Portulaca oleracea L.), slender amaranth (Amaranthus viridis L.), sicklepod (Cassia obtusifolia), black nightshade (Solanum nigrum L.), pale smartweed (Polygonum lapathifolium L.), common chickweed (Stellaria media L.), common cocklebur (Xanthium strumarium L.), flexuous bittercress (Cardamine flexuosa WITH.), henbit (Lamium amplexicaule L.) and threeseeded copperleaf (Acalypha australis L.). Accordingly, it is useful for controlling noxious weeds non-selectively or selectively in the cultivation of a crop plant such as corn (Zea mays L.), soybean (Glycine max Merr.), cotton (Gossypium spp.), wheat (Triticum spp.), rice (Oryza sativa L.), barley (Hordeum vulgare L.), oat (Avena sativa L.), sorghum (Sorghum bicolor Moench), canola (Brassica napus L.), sunflower (Helianthus annuus L.), sugar beet (Beta vulgaris L.), sugar cane (Saccharum officinarum L.), Japanese lawngrass (Zoysia Japonica stend), peanut (Arachis hypogaea L.) or flax (Linum usitatissimum L.).

For use as herbicides, the active ingredients of this invention are formulated into herbicidal compositions by mixing herbicidally active amounts with inert ingredients known to the art to facilitate either the suspension, dissolution or emulsification of the active ingredient for the desired use. The type of formulation prepared recognizes the facts that formulation, crop and use pattern all can influence the activity and utility of the active ingredient in a particular use. Thus for agricultural use the present herbicidal compounds may be formulated as water dispersible granules, granules for direct application to soils, water soluble concentrates, wettable powders, dusts, solutions, emulsifiable concentrates (EC), microemulsion, suspoemulsion, invert emulsion or other types of formulations, depending on the desired weed targets, crops and application methods.

These herbicidal formulations may be applied to the target area (where suppression of unwanted vegetation is the objective) as dusts, granules or water or solvent diluted sprays. These formulation may contain as little as 0.1% to as much as 97% active ingredient by weight.

Dusts are admixtures of the active ingredient with finely ground materials such as clays (some examples include kaolin and montmorillonite clays), talc, granite dust or other organic or inorganic solids which act as dispersants and carriers for the active ingredient; these finely ground materials have an average particle size of less than 50 microns. A typical dust formulation will contain 1% active ingredient and 99% carrier.

Wettable powders are composed of finely ground particles which disperse rapidly in water or other spray carriers. Typical carriers include kaolin clays, Fullers earth, silicas and other absorbent, wettable inorganic materials. Wettable powders can be prepared to contain from 1 to 90% active ingredient, depending on the desired use pattern and the absorbability of the carrier. Wettable powders typically contain wetting or dispersing agents to assist dispersion in water or other carriers.

Water dispersible granules are granulated solids that freely disperse when mixed in water. This formulation typically consists of the active ingredient (0.1% to 95% active ingredient), a wetting agent (1-15% by weight), a dispersing agent (1 to 15% by weight) and an inert carrier (1-95% by weight). Water dispersible granules can be formed by mixing the ingredients intimately then adding a small amount of water on a rotating disc (said mechanism is commercially available) and collecting the agglomerated granules. Alternatively, the mixture of ingredients may be mixed with an optimal amount of liquid (water or other liquid) and passed through an extruder (said mechanism is commercially available) equipped with passages which allow for the formation of small extruded granules. Alternatively, the mixture of ingredients can be granulated using a high speed mixer (said mechanism is commercially available) by adding a small amount of liquid and mixing at high speeds to affect agglomeration. Alternatively, the mixture of ingredients can be dispersed in water and dried by spraying the dispersion through a heated nozzle in a process known as spray drying (spray drying equipment is commercially available). After granulation the moisture content of granules is adjusted to an optimal level (generally less than 5%) and the product is sized to the desired mesh size.

Granules are granulated solids that do not disperse readily in water, but instead maintain their physical structure when applied to the soil using a dry granule applicator. These granulated solids may be made of clay, vegetable material such as corn cob grits, agglomerated silicas or other agglomerated organic or inorganic materials or compounds such as calcium sulfate. The formulation typically consists of the active ingredient (1 to 20%) dispersed on or absorbed into the granule. The granule may be produced by intimately mixing the active ingredient with the granules with or without a sticking agent to facilitate adhesion of the active ingredient to the granule surface, or by dissolving the active ingredient in a solvent, spraying the dissolved active ingredient and solvent onto the granule then drying to remove the solvent. Granular formulations are useful where in-furrow or banded application is desired.

Emulsifiable concentrates (EC) are homogeneous liquids composed of a solvent or mixture of solvents such as xylenes, heavy aromatic naphthas, isophorone or other proprietary commercial compositions derived from petroleum distillates, the active ingredient and an emulsifying agent or agents. For herbicidal use, the EC is added to water (or other spray carrier) and applied as a spray to the target area. The composition of an EC formulation can contain 0.1% to 95% active ingredient, 5 to 95% solvent or solvent mixture and 1 to 20% emulsifying agent or mixture of emulsifying agents.

Suspension concentrate (also known as flowable) formulations are liquid formulations consisting of a finely ground suspension of the active ingredient in a carrier, typically water or a non-aqueous carrier such as an oil. Suspension concentrates typically contain the active ingredient (5 to 50% by weight), carrier, wetting agent, dispersing agent, anti-freeze, viscosity modifiers and pH modifiers. For application, suspension concentrates are typically diluted with water and sprayed on the target area.

Solution concentrates are solutions of the active ingredient (1 to 70%) in solvents which have sufficient solvency to dissolve the desired amount of active ingredient. Because they are simple solutions without other inert ingredients such as wetting agents, additional additives are usually added to the spray tank mix before spraying to facilitate proper application.

Microemulsions are solutions consisting of the active ingredient (1 to 30%) dissolved in a surfactant or emulsifier, with additional solvents. Microemulsions are particularly useful when a low odor formulation is required such as in residential turfgrass applications.

Suspoemulsions are combinations of two active ingredients. One active ingredient is made as a suspension concentrate (1-50% active ingredient) and the second active is made as a emulsifiable concentrate (0.1 to 20%). A reason for making this kind of formulation is the inability to make an EC formulation of the first ingredient due to poor solubility in organic solvents. The suspoemulsion formulation allows for the combination of the two active ingredients to be packaged in one container, thereby minimizing packaging waste and giving greater convenience to the product user.

The herbicidal compounds of this invention may be formulated or applied with insecticides, fungicides, acaricides, nematicides, fertilizers, plant growth regulators or other agricultural chemicals. Certain tank mix additives, such as spreader stickers, penetration aids, wetting agents, surfactants, emulsifiers, humectants and UV protectants may be added in amounts of 0.01% to 5% to enhance the biological activity, stability, wetting, spreading on foliage or uptake of the active ingredients on the target area or to improve the suspensibility, dispersion, redispersion, emulsifiability, UV stability or other physical or physico-chemical property of the active ingredient in the spray tank, spray system or target area.

The compositions of the present invention may be used in admixture with or in combination with other agricultural chemicals, fertilizers, adjuvants, surfactants, emulsifiers, oils, polymers or phytotoxicity-reducing agents such as herbicide safeners. In such a case, they may exhibit even better effects or activities. As other agricultural chemicals, herbicides, fungicides, antibiotics, plant hormones, plant growth regulators, insecticides, or acaricides may, for example, be mentioned. Especially with herbicidal compositions having the compounds of the present invention used in admixture with or in combination with one or more active ingredients of other herbicides, it is possible to improve the herbicidal activities, the range of application time(s) and the range of applicable weed types. Further, the compounds of the present invention and an active ingredient of another herbicide may be separately formulated so they may be mixed for use at the time of application, or both may be formulated together. The present invention covers such herbicidal compositions.

The blend ratio of the compounds of the present invention with the active ingredient of other herbicides can not generally be defined, since it varies depending on the time and method of application, weather conditions, soil type and type of formulation. However one active ingredient of other herbicide may be incorporated usually in an amount of 0.01 to 100 parts by weight, per one part by weight of the compounds of the present invention. Further, the total dose of all of the active ingredients is usually from 1 to 10000 g/ha, preferably from 5 to 500 g/ha. The present invention covers such herbicidal compositions.

As the active ingredients of other herbicides, the following (common name) may be mentioned. Herbicidal compositions having the compounds of the present invention used in combination with other herbicides, may occasionally exhibit a synergistic effect.

1. Those that are believed to exhibit herbicidal effects by disturbing auxin activities of plants, including a phenoxy acetic acid type such as 2,4-D, 2,4-DB, 2,4-DP, MCPA, MCPP, MCPB or naproanilide (including the free acids, esters or salts thereof), an aromatic carboxylic type such as 2,3,6 TBA, dicamba, dichlobenil, a pyridine type such as picloram (including free acids and salts thereof), triclopyr or clopyralid and others such as naptalam, benazolin, quinclorac, quinmerac or diflufenzopyr (BAS 654H).

2. Those that are believed to exhibit herbicidal effects by inhibiting photosynthesis of plants including a urea type such as diuron, linuron, isoproturon, chlorotoluron, metobenzuron, tebuthiuron or fluometuron, a triazine type such as simazine, atrazine, cyanazine, terbuthylazine, atraton, hexazinone, metribuzin, simetryn, ametryn, prometryn, dimethametryn or triaziflam, a uracil type such as bromacil, terbacil or lenacil, an anilide type such as propanil or cypromid, a carbamate type such as desmedipham or phenmedipham, a hydroxybenzonitrile type such as bromoxynil or ioxynil, and others such as pyridate, bentazon and methazole.

3. A quaternary ammonium salt type such as paraquat, diquat or difenzoquat, which is believed to form active oxygen in the plant and thus to exhibit quick herbicidal effects.

4. Those which are believed to exhibit herbicidal effects by inhibiting chlorophyll biosynthesis in plants and abnormally accumulating a photsensitizing peroxide substance in the plant body, including a diphenyl ether type such as nitrofen, lactofen, acifluorfen-sodium, oxyfluorfen, fomesafen, bifenox, or chlomethoxyfen, a cyclic imide type such as chlorphthalim, flumioxazin, cinidon-ethyl, or flumiclorac-pentyl, and others such as oxadiazon, sulfentrazone, thidiazimin, azafenidin, carfentrazone, isopropazole, fluthiacet-methyl, pentoxazone, pyraflufen-ethyl and oxadiargyl.

5. Those which are believed to exhibit herbicidal effects characterized by whitening activities by inhibiting chromogenesis of plants such as carotenoids including a pyridazinone type such as norflurazon, chloridazon or metflurazon, a pyrazol type such as pyrazolate, pyrazoxyfen or benzofenap, and others such as fluridone, fluramone, diflufencam, methoxyphenone, clomazone, amitrole, sulcotrione, mesotrione, isoxaflutole and isoxachlortole.

6. Those which exhibit herbicidal effects specifically to gramineous plants including an aryloxyphenoxypropionic acid type (either as a mixture of isomers or as a resolved isomer) such as diclofop-methyl, pyrofenop-sodium, fluazifop butyl or fluazifop-p-butyl, haloxyfop-methyl, quizalofop p-ethyl, quizalafop p-tefuryl, fenoxaprop ethyl or fenoxaprop-p-ethyl, flamprop-M-methyl or flamprop-m-isopropyl or cyhalofop-butyl and a cyclohexanedione type such as alloxydim-sodium, sethoxydim, clethodim, tepraloxydim or tralkoxydim.

7. Those which are believed to exhibit herbicidal effects by inhibiting amino acid biosynthesis of plants, including a sulfonylurea type such as chlorimuron-ethyl, nicosulfuron, metsulfuron-methyl, triasulfuron, primisulfuron, tribenuron-methyl, chlorosulfuron, bensulfuron-methyl, sulfometuron-methyl, prosulfuron, halosulfuron or halosulfuron-methyl, thifensulfuron-methyl, rimsulfuron, azimsulfuron, flazasulfuron, imazosulfuron, cyclosulfamuron, flupyrsulfuron, iodosulfuron, ethoxysulfuron, flucarbazone, sulfosulfuron, oxasulfuron a triazolopyrimidinesulfonamide type such as flumetsulam, metosulam, chloransulam or chloransulam-methyl, an imidazolinone type such as imazapyr, imazethapyr, imazaquin, imazamox, imazameth, imazamethabenz methyl, a pyrimidinesalicylic acid type such as pyrthiobac-sodium, bispyribac-sodium, pyriminobac-methyl or pyribenzoxim (LGC-40863), and others such as glyphosate, glyphosate-ammonium, glyphosate-isopropylamine or sulfosate.

8. Those which are believed to exhibit herbicidal effects by interfering with the normal metabolism of inorganic nitrogen assimilation such as glufosinate, glufosinate-ammonium, phosphinothricin or bialophos.

9. Those which are believed to exhibit herbicidal effects by inhibiting cell division of plant cells, including a dinitroaniline type such as trifluralin, oryzalin, nitralin, pendamethalin, ethafluralin, benefin and prodiamine, an amide type such as bensulide, napronamide, and pronamide, a carbamate type such as propham, chlorpropham, barban, and asulam, an organophosphorous type such as amiprofos-methyl or butamifos and others such as DCPA and dithiopyr.

10. Those which are believed to exhibit herbicidal effects by inhibiting protein synthesis of plant cells, including a chloroacetanilide type such as alachlor, metolachor (including combinations with safeners such as benoxacor, or resolved isomeric mixtures of metolachlor including safeners such as benoxacor) propachlor, acetochlor (including combinations with herbicide safeners such as dichlormid or MON 4660 or resolved isomeric mixtures of acetochlor containing safeners such as dichlormid or MON 4660), propisochlor or dimethenamid or an oxyacetamide type such as flufenacet.

11. Those in which the mode of action causing the herbicidal effects are not well understood including the dithiocarbamates such as thiobencarb, EPTC, diallate, triallate, molinate, pebulate, cycloate, butylate, vernolate or prosulfocarb and miscellaneous herbicides such as MSMA, DSMA, endothall, ethofumesate, sodium chlorate, pelargonic acid and fosamine.

A few formulation examples of the present invention are given as follows.

| | | | | |
|---|---|---|---|---|
| Ingredient | Chemical | | | % |
| Trade Name | Name | Supplier | Function | wt./wt. |

Formulation example 1. Emulsifiable Concentrate

| | | | | |
|---|---|---|---|---|
| Compound 1-1 | | | Active | 5.0 |
| | | | Ingredient | |
| Toximul H-A | Calcium | Stepan Co. | Emulsifier | 2.5 |
| | sulfonate and | | | |
| | nonionic | | | |
| | surfactant | | | |
| | blend | | | |
| Toximul D-A | Calcium | Stepan Co. | Emulsifier | 7.5 |
| | sulfonate and | | | |
| | nonionic | | | |
| | surfactant | | | |
| | blend | | | |
| Aromatic 200 | Aromatic | Exxon | Solvent | QS to |
| | hydrocarbon | Chemical Co. | 100% | |

Formulation example 2. Suspension Concentrate

| | | | | |
|---|---|---|---|---|
| Compound 1-1 | | | Active | 10.00 |
| | | | Ingredient | |
| Proylene gylcol | | | Anti-freeze | 5.00 |
| Antifoam 1530 | Silicone | Dow Corning | Anti-foam | 0.50 |
| | defoamer | | | |
| Rhodopol 23 | Xanthan gum | Rhone-Poulene | Suspending | 0.25 |
| | | | Aid | |
| Morwet D-425 | Napthalene | Witco Corp. | Dispersant | 3.00 |
| | formaldehyde | | | |
| | condensate | | | |
| Igepal CA-720 | Octylphenol | Rhone-Poulene | Wetting | 3.00 |
| | ethoxylate | | agent | |
| Proxel GXL | 1,2 benziso- | ICI Americas | Preservative | 0.25 |
| | thiazolin-3-one | | | |
| Water | | | Diluent | 68.00 |

Formulation example 3. Wettable Powder

| | | | | |
|---|---|---|---|---|
| Compound 1-1 | | | Active | 50.00 |
| | | | Ingredient | |
| Geropon T-77 | Sodium-N- | Rhone-Poulene | Wetting | 3.00 |
| | methyl-N- | | agent | |
| | oleoyl | | | |
| | taurate | | | |
| Lomar PW | Napthalene | Henkel Corp. | Dispersant | 5.00 |
| | Sulfonate | | | |
| Kaolin clay | Kaolin clay | J. M. Huber | Filler | 42.00 |

Formulation example 4. Water Dispersible Granule

| | | | | |
|---|---|---|---|---|
| Compound 1-1 | | | Active | 50.00 |
| | | | Ingredient | |
| Morwet EFW | | Witco Corp. | Wetting | 2.00 |
| | | | agent | |
| Morwet D-425 | Napthalene | Witco Corp. | Dispersant | 10.00 |
| | formaldehyde | | | |
| | condensate | | | |
| ASP 400 | Kaolin Clay | Engelhard | Filler | 38.00 |
| | | Corp. | | |

### TEST EXAMPLE

A standard greenhouse herbicide activity screening system was used to evaluate the herbicidal efficacy and crop safety of these test compounds. Seven broadleaf weed species including redroot pigweed (Amaranthus retroflexus, AMARE), velvetleaf (Abutilon theophrasti, ABUTH), sicklepod (Cassia obtusifolia, CASOB), ivyleaf morningglory (Ipomoea hederacea, IPOHE), lambsquarters (Chenopodium album, CHEAL), common ragweed (Ambrosia artemisiifolia L., AMBEL), and cocklebur (Xanthium strumarium, XANST) were used as test species. Four grass weed species including green foxtail (Setaria viridis, SETVI), barnyardgrass (Echinochloa crus-galli, ECHCG), johnsongrass (Sorghum halepense, SORHA), and large crabgrass (Digitaria sanguinalis, DIGSA) were also used. In addition, three crop species, field corn (Zea mays L., var. Dekalb 527, CORN), soybean (Glycine max L., var. Pella 86, SOY), and upland rice (Oryza sp., var. Tebonnet, RICE) were included.

Pre-Emerge Test

All plants were grown in 10 cm square plastic pots which were filled with a sandy loam soil mix. For pre-emerge tests, seeds were planted one day prior to application of the test compounds. For post-emerge tests, seeds were planted 8-21 days prior to the test to allow emergence and good foliage development prior to application of the test substances. At the time of the post-emerge application, plants of all species were usually at the 2-3 leaf stage of development.

All test compounds were dissolved in acetone and applied to the test units in a volume of 187 l/ha. Test materials were applied at rates ranging from 15 g ai/ha to 1000 g ai/ha using a track sprayer equipped with a TJ8001 E even flow flat fan spray nozzle. Plants were arranged on a shelf so that the top of the canopy (post-emerge) or top of the soil surface (pre-emerge) was 40-45 cm below the nozzle. Pressurized air was used to force the test solution through the nozzle as it was mechanically advanced over the top of all test plants/pots. This application simulates a typical commercial field herbicide application.

Post-Emerge Test

In the post-emerge test, a commercial non-ionic surfactant was also included (0.25% v/v) to enhance wetting of the leaf surfaces of target plants. Immediately after application, test units of the pre-emerge applications were watered at the soil surface to incorporate the test materials.

At 14 days after application of the test materials, phytotoxicity ratings were recorded. A rating scale of 0-100 was used as previously described in Research Methods in Weed Science, 2nd edition, B. Truelove, Ed., Southern Weed Science Society, Auburn University, Auburn, Ala., 1977. Briefly, “0” corresponds to no damage and “100” corresponds to complete death of all plants in the test unit. This scale was used both to determine efficacy against weed species and damage to crop species. Herbicide activity data for various compounds of this invention, which are shown by compound No. in Tables 1-7, are shown in Tables 8 and 9. The data demonstrate significant differences between compounds for both efficacy against weeds and selectivity for crop species. For selected compounds, excellent activity against a majority of the weed species was observed with minimal damage to at least one of the crop species.

Tables 8 and 9 show pre-emerge and post-emerge herbicidal activity data respectively for a few representative examples of the compounds described herein.

TABLE 8
Pre-emerge Herbicidal Activity

| Compd. | Rate g | | | | | | | | | | | | | |
|---|---|---|---|---|---|---|---|---|---|---|---|---|---|---|
| No. | al/ha | AMARE | ABUTH | CASOB | IPOHE | CHEAL | AMBEL | SETVI | ECHCG | SORHA | DIGSA | CORN | SOY | RICE |
| 1-1 | 63 | 100 | 100 | 100 | 100 | 100 | 100 | 100 | 100 | 100 | 100 | 100 | 100 | 90 |
| 1-1 | 250 | 100 | 100 | 100 | 100 | 100 | 100 | 100 | 100 | 100 100 | 100 | 100 | 99 | |
| 1-2 | 63 | 100 | 100 | 100 | 100 | 100 | 100 | 100 | 100 | 100 100 | 100 | 100 | 99 | |
| 1-2 | 250 | 100 | 100 | 100 | 100 | 100 | 100 | 100 | 100 | 100 100 | 100 | 100 | 100 | |
| 1-3 | 63 | 100 | 100 | 95 | 95 | 100 | 99 | 100 | 100 | 100 | 100 | 50 | 40 | 70 |
| 1-3 | 250 | 100 | 100 | 100 | 100 | 100 | 100 | 100 | 100 | 100 100 | 95 | 99 | 80 | |
| 1-4 | 63 | 100 | 100 | 70 | 99 | 100 | 80 | 100 | 100 | 100 | 100 | 15 | 10 | 60 |
| 1-4 | 250 | 100 | 100 | 100 | 100 | 100 | 100 | 100 | 100 | 100 100 | 85 | 80 | 90 | |
| 1-5 | 63 | 100 | 100 | 90 | 100 | 100 | 99 | 100 | 100 | 99 | 100 | 85 | 0 | 55 |
| 1-5 | 250 | 100 | 100 | 100 | 100 | 100 | 100 | 100 100 | 100 | 100 | 100 | 99 | 80 | |
| 2-2 | 63 | 100 | 100 | 99 | 99 | 100 | 100 | 100 | 99 | 100 | 100 | 50 | 50 | 60 |
| 2-2 | 250 | 100 | 100 | 100 | 99 | 100 | 100 | 100 | 100 | 100 | 100 | 98 | 95 | 75 |

TABLE 9
Post-emerge Herbicidal Activity

| Compd. | Rate g | | | | | | | | | | | | | |
|---|---|---|---|---|---|---|---|---|---|---|---|---|---|---|
| No. | al/ha | AMARE | ABUTH | CASOB | IPOHE | CHEAL | AMBEL | SETVI | ECHCG | SORHA | DIGSA | CORN | SOY | RICE |
| 1-1 | 63 | 100 | 100 | 100 | 100 | 100 | 100 | 100 | 100 | 95 | 95 | 12 | 100 | 80 |
| 1-1 | 250 | 100 | 100 | 100 | 100 | 100 | 100 | 100 | 100 | 100 | 99 | 40 | 100 | 90 |
| 1-2 | 63 | 100 | 100 | 100 | 100 | 100 | 100 | 100 | 100 | 100 | 99 | 25 | 100 | 75 |
| 1-2 | 250 | 100 | 100 | 100 | 100 | 100 | 100 | 100 | 100 | 100 100 | 35 | 100 | 80 | |
| 1-3 | 63 | 100 | 100 | 70 | 100 | 100 | 99 | 99 | 100 | 65 | 50 | 15 | 75 | 90 |
| 1-3 | 250 | 100 | 100 | 100 | 100 | 100 | 100 | 100 | 100 | 85 | 65 | 15 | 99 | 99 |
| 1-4 | 63 | 100 | 100 | 60 | 95 | 100 | 90 | 50 | 90 | 20 | 0 | 0 | 50 | 60 |
| 1-4 | 250 | 100 100 | 99 | 100 | 100 | 90 | 100 | 100 | 30 | 20 | 30 | 50 | 65 | |
| 1-5 | 63 | 99 | 100 | 60 | 100 | 95 | 90 | 80 | 100 | 55 | 60 | 15 | 50 | 65 |
| 1-5 | 250 | 100 | 100 | 90 | 100 | 100 | 100 | 85 | 100 | 75 | 75 | 30 | 75 | 70 |
| 2-2 | 63 | 100 | 100 | 100 | 100 | 100 | 100 | 100 | 100 | 100 | 85 | 40 | 100 | 50 |
| 2-2 | 250 | 100 | 100 | 100 | 100 | 100 | 100 | 100 | 100 | 100 | 100 | 45 | 100 | 70 |

What is claimed is:

1. A compound represented by the formula (1) or its salt 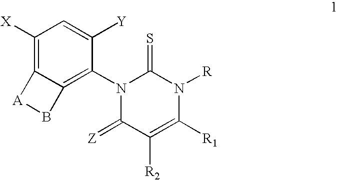
wherein
X is halogen, cyano, nitro, or haloalkyl;
Y is hydrogen or halogen;
Z is oxygen, sulfur or imino;
R is alkyl, haloalkyl or amino;
R₁ is haloalkyl;
R₂ is hydrogen, halogen, nitro, or substituted or unsubstituted amino group;
A is —N═C(R₃)—; —C(R₃)═C(R₄)—; —N(R₃)—C(R₃′)=C(R₄)—; —O—C(R₃)(R₄)—C(R₃′)(R₄′)-; —O—C(R₃)═C(R₄)—; or —C(R₃)═C(R₃′)-C(R₃″)(R₄)—;
B is oxygen or a bond; and
R₃ and R₄ can be taken together to represent oxygen, sulfur or an unsubstituted or substituted imino or an oxime group; or R₃, R₃′, R₃″, R₄ and R₄′ are independent of each other and is selected from the group consisting of hydrogen, halogen, hydroxy, mercapto, amino, cyano, nitro, (C1-6)alkyl, (C1-6)haloalkyl, (C1-6)alkoxy,(C1-6)haloalkoxy, (C1-6)alkoxyalkyl, (C2-6)alkynyl, (C2-6)alkenyl, aryl, heteroaryl, aryloxy, heteroaryloxy, (C3-6)cycloalkyl, (C3-6)cycloalkylcarbonyl, carboxy, (C1-6)alkylcarbonyl, arylcarbonyl, (C1-3)haloalkylcarbonyl, (Cl-6)alkylcarbonyloxy, (C1-6)haloalkylcarbonyloxy, (C1-6)alkoxycarbonyl, (Cl-6)haloalkoxycarbonyl, (C1-6)alkylthiocarbonyl, (C1-6)haloalkylthiocarbonyl, (Cl-6)alkoxythiocarbonyl, (C1-6)haloalkoxythiocarbonyl,(C1-6)alkylamino, arylsulfonylamino, arylamino, (C1-6)alkylthio, arylthio, (C2-6)alkenylthio, (C2-6)alkynylthio, (C1-6)alkylsulfinyl, (C2-6)alkenylsulfinyl, (C2-6)alkynylsulfinyl, (C1-6)alkylsulfonyl, (C2-6)alkenylsulfonyl, (C2-6)alkynylsulfonyl, arylsulfonyl, where any of these groups may be unsubstituted or substituted with any of the functional groups represented by one more of the following; halogen, hydroxy, mercapto, cyano, nitro, amino, caboxy, (C1-6)alkyl, (C1-6)haloalkyl, (C1-6)alkylcarbonyl, (Cl-6)alkylcarbonyloxy, (C1-6)haloalkylcarbonyl, (C1-6)haloalkylcarbonyloxy, (C1-6)alkoxy, (C1-6)alkoxycarbonyl, aminocarbonyl, (C1-6)alkylaminocarbonyl, (C1-6)haloalkoxy, (C1-6)haloalkoxycarbonyl, (C1-6)alkylsulfonyl, (C1-6)haloalkylsulfonyl, aryl, halooaryl, alkoxyaryl, aryloxy, arylthio, haloaryloxy, heteroaryl, heteroaryloxy, (C3-7)cycloalkyl and other related groups.

2. The compound or its salt according to claim 1, wherein
X is halogen or cyano;
Y is a halogen;
Z is oxygen;
R is (C1-4)alkyl;
R₁ is (C1-4)haloalkyl; and
R₂ is hydrogen.

3. The compound or its salt according to claim 1, wherein
X is chlorine;
Y is fluorine;
Z is oxygen;
R is methyl;
R₁ is trifluoromethyl; and
R₂ is hydrogen.

4. The compound or its salt according to claim 1, wherein
X is chlorine;
Y is fluorine;
Z is oxygen;
R is methyl;
R₁ is trifluoromethyl;
R₂ is hydrogen; and
-A-B- is —N═C(R₃)—O—; —C(R₃)═C(R₄)—O—; —N(R₃)—C(R₃′)=C(R₄)—; —O—C(R₃)(R₄)—C(R₃′)(R₄′)-O—; —O—C(R₃)═C(R₄)—; or —C(R₃)═C(R₃′)-C(R₃″)(R₄)—O—.

5. The compound or its salt according to claim 1, wherein
X is chlorine;
Y is fluorine;
Z is oxygen;
R is methyl;
R₁ is trifluoromethyl;
R₂ is hydrogen;
-A-B- is —N═C(R₃)—O—; or —O—C(R₃)═C(R₄)—; and
R₃ and R₄ are independent of each other and may be selected from the group consisting of hydrogen, halogen, cyano, nitro, (C1-6)alkyl, (C1-6)haloalkyl, (C1-6)alkoxy,(C1-6)haloalkoxy, (C1-6)alkoxyalkyl, (C2-6)alkynyl,(C2-6)alkenyl, aryl, heteroaryl, aryloxy, heteroaryloxy, (C3-6)cycloalkyl, (C3-6)cycloalkylcarbonyl, carboxy, (C1-6)alkylcarbonyl, arylcarbonyl, (Cl-3)haloalkylcarbonyl, (C1-6)alkylcarbonyloxy, (C1-6)haloalkylcarbonyloxy, (Cl-6)alkoxycarbonyl, (C1-6)haloalkoxycarbonyl, (C1-6)alkylthiocarbonyl, (C1-6)haloalkylthiocarbonyl, (C1-6)alkoxythiocarbonyl, (C1-6)haloalkoxythiocarbonyl,(C1-6)alkylamino, arylsulfonylamino, arylamino, where any of these groups may be unsubstituted or substituted with any of the functional groups represented by one more of the following; halogen, cyano, nitro, (C1-6)alkyl, (C1-6)haloalkyl, (C1-6)alkylcarbonyl, (C1-6)alkylcarbonyloxy, (C1-6)haloalkylcarbonyl, (C1-6)haloalkylcarbonyloxy, (C1-6)alkoxy, (C1-6)alkoxycarbonyl, aminocarbonyl, (C1-6)alkylaminocarbonyl, (C1-6)haloalkoxy, (C1-6)haloalkoxycarbonyl, (C1-6)alkylsulfonyl, (C1-6)haloalkylsulfonyl, aryl, halooaryl, alkoxyaryl, aryloxy, arylthio, haloaryloxy, heteroaryl, heteroaryloxy, (C3-7)cycloalkyl and other related groups.

6. A process for producing a compound represented by the formula (1′) or its salt, 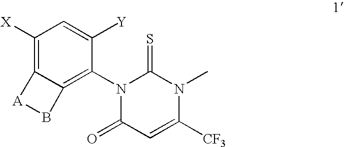
wherein X, Y, A, and B are as defined in claim 1, by the cyclization reaction of a compound represented by formula (4).

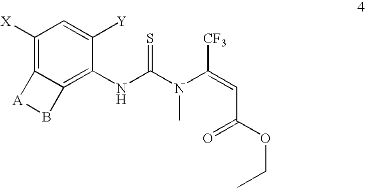

7. A process for producing a compound represented by the formula (1-a) or its salt, 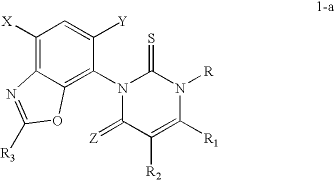
wherein X, Y, R, R₁, R₂, R₃ and Z arc as defined in claim 1 which comprises reacting a compound represented by the formula (5) 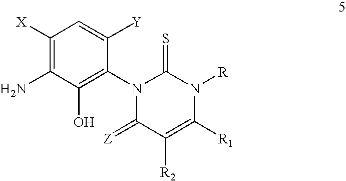
with a substituted or unsubstituted acid chloride, an ester, or an equivalent.

8. A herbicidal composition which comprises an effective amount of a compound of claim 1 and an agricultural adjuvant.

9. A method for controlling weeds, which comprises applying to the locus to be protected a herbicidally effective amount of a compound of claim 1.

10. A method according to claim 9, wherein the locus to which the compound is applied is a cornfield.

11. A method according to claim 9, wherein the locus to which the compound is applied is a soybean field.

12. A method for controlling weeds, which comprises applying to the locus to be protected a herbicidally effective amount of a compound of claim 1 in combination with another herbicide for providing an additive or synergistic herbicidal effect.

13. A method for controlling weeds of claim 12, wherein the compound of claim 1 is applied to soil as a preemergent herbicide.

14. A method for controlling weeds of claim 12, wherein the compound of claim 1 is applied to plant foliage.

15. A method for controlling weeds of claim 12, wherein the another herbicide is an acetanilide, sulfonylurea, or any referenced in the text.

16. A method to desiccate a plant which comprises applying to the plant a compound of claim 1.

17. A method according to claim 16, wherein the plant to which the compound is applied is a potato plant or a cotton plant.
